(19) United States
(12) Plant Patent Application Publication
(10) Pub. No.: US 2013/0305419 P1
(43) Pub. Date: Nov. 14, 2013

(54) Coleus plant named 'H67'

(50) ;

(71) Applicant: Robert H. Bors, Saskatoon (CA)

(72) Inventor: Robert H. Bors, Saskatoon (CA)

(73) Assignee: University of Saskatchewan, Saskatoon (CA)

(21) Appl. No.: 13/506,718

(22) Filed: May 10, 2012

(51) Int. Cl. A01H 5/00

(52) U.S. Cl. PLT/263.1

(57) ABSTRACT

A Coleus plant particularly distinguished by triangular leaves with many deep lobes and yellow-green leaves with purple margins, is disclosed.

### GENUS AND SPECIES

Solenostemon sp.

### VARIETY DENOMINATION

‘H67’

### BACKGROUND OF THE NEW PLANT

The present invention comprises a new and distinct variety of Coleus, botanically known as Solenostemon sp., and hereinafter referred to by the variety name ‘H67’. The new variety originated from an open pollination conducted in Saskatoon, Saskatchewan, Canada in the winter 2007-2008. ‘H67’ is the result of 4 generations of open pollination descending from ‘Coleus#3’ (unpatented) with ‘Coleus#21’ (unpatented). The seeds produced by the open pollination were sown in April 2008. A single plant selection was chosen for further evaluation and asexual propagation in December 2008.

The new plant was first asexually propagated in December 2008 in Saskatoon, Saskatchewan, Canada and has been asexually reproduced by vegetative cuttings for almost three years in Saskatoon, Saskatchewan, Canada. The present invention has been found to retain its distinctive characteristics through successive asexual propagations by vegetative cuttings.

‘H67’ has not been made publicly available or sold more than one year prior to the filing date of this application.

### SUMMARY OF THE NEW PLANT

The following are the most outstanding and distinguishing characteristics of the new variety when grown under normal horticultural practices in Saskatoon, Saskatchewan, Canada and Litchfield, Mich.

1. Triangular shaped leaves with many deep lobes and

2. Leaves larger than ‘Kiwi Fern’ but smaller than most coleus and

3. Yellow-green leaves with purple margins and

4. Under sunny conditions more purple appears in the center of the leaves.

### DESCRIPTION OF THE PHOTOGRAPHS

This new Coleus plant is illustrated by the accompanying photographs which show the overall plant habit, and foliage of the plant. The colors are as true as can be reasonably obtained by conventional photographic procedures. The photographs are of a 4.5 month-old plant grown in a greenhouse in Litchfield, Mich. The photographs were taken in November 2011.

FIG. 1 shows the whole plant, including habit and foliage.

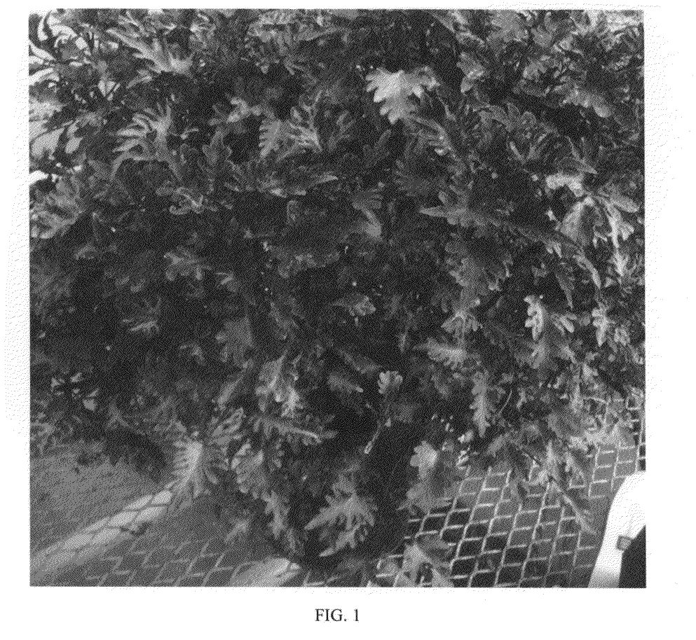

FIG. 2 shows a close-up of the foliage.

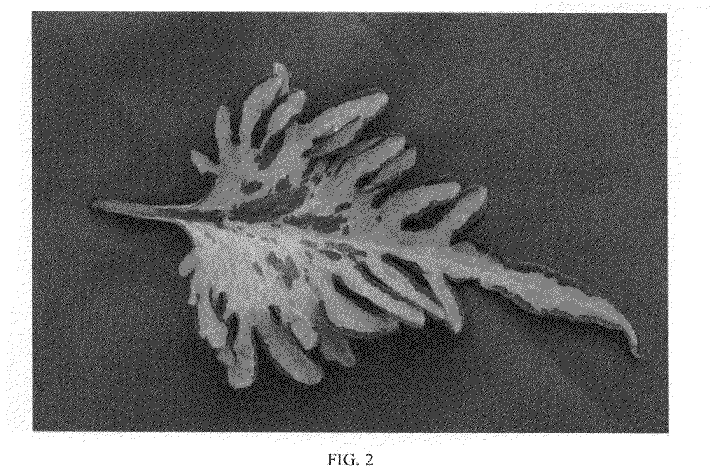

### DESCRIPTION OF THE NEW VARIETY

The following detailed descriptions set forth the distinctive characteristics of ‘H67’. The data which defines these characteristics were collected from asexual reproductions carried out in Saskatoon, Saskatchewan, Canada. The plant history was taken in October 2011 on 16-week old plants grown in a greenhouse in Litchfield, Mich. Color references were taken under natural light and are to the R.H.S. Colour Chart of The Royal Horticultural Society of London (R.H.S.), fifth edition (2007).
Classification:
   Family.—Lamiaceae.
   Botanical name.—Solenostemon sp.
   Common name.—Coleus.
   Denomination.—‘H67’.
Parentage: ‘H67’ is the result of 4 generations of open pollination descending from ‘Coleus#3’ (unpatented) with ‘Coleus#21’ (unpatented)
Plant:
   Type.—Annual.
   Habit.—Upright, basal-branched.
   Height (measured from the top of the soil).—40.6 cm.
   Width (horizontal plant diameter).—55.9 cm.
   Time to produce a finished plant.—About 6 to 8 weeks.
   Time to initiate and develop roots.—About 3 weeks.
   Root description and habit.—White roots, fibrous, and branching.
   Propagation type.—Vegetative cuttings.
Branches:
   Quantity per plant.—6 primary branches; multiple secondary branches.
   Length.—35.6 cm.
   Internode length.—1.3 cm to 3.8 cm.
   Diameter (from midpoint).—0.6 cm.
   Color.—RHS N77A (Purple).
   Petiole.—Color: RHS N77A (Purple) Length: 3.8 cm Diameter: 0.2 cm.
Leaves:
   Quantity of leaves per branch.—About 16 to 20.
   Arrangement.—Opposite/Sub-opposite.
   Length.—5.1 cm to 6.4 cm.
   Width.—2.5 cm to 4.4 cm.
   Color, immature.—Upper surface: RHS 146B (Yellow-green) and RHS N77A (Purple) on the margins Lower surface: RHS N77A (Purple).
   Color, mature.—Upper surface: RHS 146B (Yellow-green) and RHS N77A (Purple) on the margins Lower surface: RHS N77A (Purple).
   Texture.—Upper surface: Smooth Lower surface: Smooth.
   Venation type.—Venulate. Venation color: Upper surface: RHS 146D (Yellow-green) Lower surface: RHS 146D (Yellow-green).
Inflorescence: Single, bilabiate flowers arranged in verticallasters on terminal spikes; 5 petals Fragrance: Absent Spike: Length: 12.0 cm to 15.0 cm Diameter: 2.5 cm to 3.0 cm Quantity of verticillasters per spike: 7 flowers per node (18 to 42 nodes) Quantity per open flowers per verticillasters: Varies with age of plant. Flower bud: Length: 0.1 cm to 0.3 cm Diameter: 0.1 cm to 0.2 cm Color at tight bud: Lavender with white. Corolla: Diameter: 0.3 cm to 0.4 cm Length: 1.0 cm to 1.1 cm Petal arrangement: All 5 petals fused to form a tube Banner width: 0.3 cm Banner length: 0.4 cm Keel length: 0.7 cm to 0.8 cm Keel width: 0.2 cm to 0.3 cm. Calyx: Form: One single calyx tube formed by 5 fused sepals Length: 0.7 cm to 1.5 cm Diameter: 0.2 cm Color (both inner and outer surfaces): Dark purple Texture (both inner and outer surfaces): Slightly pubescent. Pedicels: Length: 0.4 cm Width (diameter): Less than 0.1 cm Texture: Hirsute Color: Dark purple.
Reproductive organs: Stamens: Quantity per flower: 4 Filament color: White Filament length: 0.8 cm to 0.9 cm Anther shape: Oval Anther length: Less than 0.1 cm Pollen color: White Pollen amount: Sparse. Pistils: Quantity per flower: 1 Length: 1.0 cm to 1.1 cm Stigma color: Lavender Stigma shape: Forked end Style color: White Ovary color: Black.
Disease or insect resistance: No disease or insect resistance observed.
Comparison with commercial variety ‘H67’ differs from the commercial comparison ‘Kiwi Fern’ (patent status unknown) in that ‘H67’ has yellow-green leaves with purple margins, while ‘Kiwi Fern’ has brown leaves. Additionally, ‘H67’ has larger leaves and is less branching than ‘Kiwi Fern’. ‘H67’ leaves have a triangular shape while ‘Kiwi Fern’ is more cylindrical.

I claim:

1. A new and distinct variety of Coleus plant as shown and described herein.
